(19) United States
(12) Patent Application Publication
(10) Pub. No.: US 2010/0166746 A1
(43) Pub. Date: Jul. 1, 2010

(54) HIGH POTENCY RECOMBINANT ANTIBODIES, METHODS FOR PRODUCING THEM AND USE IN CANCER THERAPY

(75) Inventors: Partha Chowdhury, Gaithersburg, MD (US); Herren Wu, Boyds, MD (US); Laura Richman, Damascus, MD (US)

Correspondence Address: MEDIMMUNE, LLC;Patrick Scott Alban, ONE MEDIMMUNE WAY, GAITHERSBURG, MD, 20878, US

(73) Assignee: Medlmmune Way, Gaithersburg, MD (US)

(21) Appl. No.: 12/517,300

(22) PCT Filed: Dec. 4, 2007
(86) PCT No.: PCT/US07/24775

§ 371 Date: Mar. 8, 2010

Related U.S. Application Data
(60) Provisional application No. 60/868,532, filed on Dec. 4, 2006.

(51) Int. Cl. A61K 39/395; C07K 16/30; A61P 35/00

(52) U.S. Cl. 424/133.1; 530/387.3

(57) ABSTRACT

The present invention contemplates improved recombinant anti-tumor antibodies having faster Kon and faster Koff rates, resulting in a uniform tumor penetrance, as compared to the same recombinant anti-tumor antibody without said faster Kon and faster Koff rates, and methods of improving the same.

### BACKGROUND OF THE INVENTION

The present invention relates to high potency antibodies, methods of increasing antibody potency and to methods of using such antibodies for prevention and treatment of diseases.

Antibodies have been, and are currently being, developed for the prevention and treatment of various diseases, such as, for example, the prevention and/or treatment of cancer. One approach has been the development of antibodies, some with high specific neutralizing activity. The production of high potency antibodies (i.e., antibodies with high biological activity, such as antigen neutralizing ability), including antibodies with ultra high affinity for the target antigen, would be desirable from the point of view of both the neutralizing ability of such an antibody as well as from the more practical aspects of requiring less antibody in order to achieve a desirable degree of clinical effectiveness, thereby cutting costs of use.

Antibody affinity is measured by the binding constant of the antibody for a particular antigen, and such binding constant is often calculated by the ratio of the rate constant for antibody-antigen complex formation (referred to as the “Kₒₙ” value) to the rate constant for dissociation of said complex (the “K_{off}” value).

Tumor penetration by antibody therapeutics is one of the challenges for antibody cancer therapy. Because slow K_{off} rates mediate the prolonged retention of antibodies on tumor cells, it has been assumed that the best tumor targeting will be achieved with the highest affinity antibodies, where the high affinity is usually driven by a low K_{off}. However, very high affinity antibodies may not always lead to good tumor penetration since the periphery of a target organ soaks up most of the antibody trying to reach the tumor interior, thereby preventing the antibody from invading the interior of the tumor. This challenge was first postulated by Weinstein, who called this phenomenon the “binding site barrier” effect in which antibodies with very high affinity (i.e., prolonged K_{off}), for tumor antigens stably bind to the first encountered tumor antigen (see Fujimori, K., et al., A Modeling Analysis Of Monoclonal Antibody Percolation Through Tumors: A Binding Site Barrier. J. Nucl. Med., 31:1191-1198 (1990)). Affinity barriers for antibodies are created when the high affinity is dictated by faster Kₒₙ and slower K_{off}. Under this situation antibodies after extra-vasation tend to bind to antigen and not come off easily. As a result they cannot progress deeper into the tumor or away from the blood vessels. This is further complicated by the outwardly directed interstitial pressure arising at the interior of the tumor. This can lead to patchy and incomplete tumor perfusion and could be associated with sub-optimal therapeutic effects when therapeutic efficacy is dependent upon uniform delivery to tumor cells. In order to effectively penetrate into a tumor an antibody must have an inward diffusional velocity greater than the outward convective velocity.

Overcoming the binding site barrier effect may not be a simple matter of administering lower affinity antibodies to a tumor—such antibodies may not exert enough cancer killing due to a low amount of antibody binding, even if the binding site barrier effect in and of itself is overcome. One way to achieve this is to reduce the size of whole antibodies by converting them into Fvs and Fab or Fab'2, see also Yokota T., et. al., Cancer Research 52, 3402-3408, Jun. 15, 1992; and Adams G., et. al., Cancer Research 61, 4750-4755, Jun. 15, 2001; and Beckman R., et. al., Cancer (2007) 109:170-179. But in so doing, the effector properties of antibodies such as antibody dependent cell-mediated cytotoxicity (ADCC) and CDC are compromised. The other way is to modulate the kinetic parameters such as the Kₒₙ and slower K_{off} rates of antibodies. It is conceivable that in order to penetrate into tumors the high affinity of an antibody should be driven by faster Kₒₙ and reasonable or medium rate K_{off}. Under this situation antibody molecules after extravasation would bind well to the target and also dissociate relatively easily to be in solution for greater period of time. This is likely to facilitate their diffusion through the extracellular fluid into deeper areas of the tumor.

In accordance with the present invention, it is believed that antibody potency, which is a function of the Kₒₙ value, irrespective of specificity, may solve the dual problems of providing an antibody that is (a) able to bind to its intended target tightly, and (b) able to overcome the binding site barrier effect, in order to achieve an effective cancer therapy. Therefore, high affinity antibodies, driven by high Kₒₙ values may be able to bind tumor antigens with a resulting better tumor penetration than those antibodies, binding the same tumor antigens, that are driven by a low K_{off} value.

A high, affinity antibody driven by high Kₒₙ values rather than low K_{off} values should reach an equilibrium binding faster, thereby allowing a sufficient quantity of antibodies binding to tumor antigens to exert an effective cancer killing. As a result, such high Kₒₙ antibodies are not expected to adhere on the peripheral tumor layer for a very long period of time because such antibodies will have K_{off} values that are not low (i.e., have K_{off} values that are high). Therefore, such antibodies are expected to dissociate from tumor antigens on the periphery more quickly, thereby enabling these antibodies to subsequently bind again to another tumor antigen located in a deeper tumor layer. This type of binding equilibrium is expected to result in better tumor penetration by the antibody and therefore, a more effective cancer killing for treatment or, in other words, more potent cancer therapeutics.

### SUMMARY OF THE INVENTION

The present invention contemplates, but is not limited to, the following embodiments:

A method of improving a recombinant anti-tumor antibody's penetrance into a targeted tumor, comprising: (a) increasing the Kₒₙ rate by at least 2 fold as compared to the same recombinant anti-tumor antibody without such an increase in Kₒₙ rate; and achieving a uniform tumor penetrance by said improved recombinant anti-tumor antibody into said targeted tumor as measured by immunohistochemistry staining (IHC) as compared to the same recombinant anti-tumor antibody without such an increase in Kₒₙ rate and demonstrates a non-uniform or patchy staining as measured by IHC. The method may further comprises after step (a), a step of increasing the K_{off} rate by at least 2 fold as compared to the same recombinant anti-tumor antibody without such an increase in K_{off} rate. Said Kₒₙ rate and said K_{off} rate are both measured by surface plasmon resonance.

In another embodiment, the aforementioned method of improving a recombinant anti-tumor antibody demonstrates a Kₒₙ rate of at least 10³ M⁻¹s⁻¹ or greater, at least 10⁵ M⁻¹s⁻¹ or greater, as measured by SPR.

In yet another embodiment, the method of the invention improves said antibody wherein said improved recombinant anti-tumor antibody has a K_{off} rate of 10⁻³ s⁻¹ or less, or 10⁻⁴ s⁻¹ or less, as measured by SPR.

In another embodiment, the method of the invention improves said antibody wherein said improved recombinant anti-tumor antibody has a K_{D} of less than 10000 pM or of between 25 to 10000 pM.

An embodiment of the invention includes the above-described method of improving a recombinant anti-tumor antibody's penetrance into a targeted tumor, wherein said uniform tumor penetrance by said improved recombinant anti-tumor antibody into said targeted tumor demonstrates at least 70%, at least 80%, at least 90% or 100% IHC staining.

Another embodiment of the invention includes a method of improving a recombinant anti-tumor antibody's penetrance into a targeted tumor, comprising: (a) increasing the Kₒₙ rate by at least 2 fold as compared to the same recombinant anti-tumor antibody without such an increase in Kₒₙ rate; and (b) achieving an intra-tumoral diffusion greater than 3 tumor cell diameters as measured by IHC, as compared to the same recombinant anti-tumor antibody without such an increase in Kₒₙ rate and demonstrates an intra-tumoral diffusion of 3 or less tumor cell diameters as measured by IHC.

Yet another embodiment of the invention includes an improved recombinant anti-tumor antibody that specifically binds to the targets CD20, HER2/neu, VEGF, EphA2, integrin αᵥβ3, EphA4, CD2, CD19, CD22, cMET, ALK, DLL4 or CEA.

In other embodiments, the improved recombinant anti-tumor antibodies are monoclonal, chimeric, humanized or human.

In one embodiment, a recombinant anti-tumor antibody having improved tumor penetrance into a targeted tumor, is produced by a method comprising: (a) increasing the Kₒₙ rate by at least 2 fold as compared to the same recombinant anti-tumor antibody without such an increase in Kₒₙ rate; and (b) achieving a uniform tumor penetrance by said improved recombinant anti-tumor antibody into said targeted tumor as measured by immunohistochemistry staining (IHC) as compared to the same recombinant anti-tumor antibody without such an increase in Kₒₙ rate and demonstrates a non-uniform or patchy staining as measured by IHC. Such an improved antibody may also have a step of increasing the K_{off} rate by at least 2 fold as compared to the same recombinant anti-tumor antibody without such an increase in K_{off} rate.

In yet another embodiment, a recombinant anti-tumor antibody having improved tumor penetrance into a targeted tumor, is produced by a method comprising: (a) increasing the Kₒₙ rate by at least 2 fold as compared to the same recombinant anti-tumor antibody without such an increase in Kₒₙ rate; and (b) achieving an intra-tumoral diffusion greater than 3 tumor cell diameters as measured by IHC, as compared to the same recombinant anti-tumor antibody without such an increase in Kₒₙ rate and demonstrates an intra-tumoral diffusion of 3 or less tumor cell diameters as measured by IHC. Such an improved antibody may also have a step of increasing the K_{off} rate by at least 2 fold as compared to the same recombinant anti-tumor antibody without such an increase in K_{off} rate.

In one embodiment, the recombinant anti-tumor antibody having improved tumor penetrance is covalently linked to a cytotoxic moiety, such as a radionucleotides, toxins or chemotherapeutic agents.

In another embodiment, a composition comprises, the recombinant anti-tumor antibody having improved tumor penetrance as described above, and a pharmacologically acceptable diluent or excipient.

In yet another embodiment, a method of treating a tumor in a patient in need thereof, comprising administering a therapeutically effective amount of the composition described above.

Embodiment 1: An isolated antibody having a Kₒₙ of at least 10⁴ M⁻¹s⁻¹ or greater, wherein said antibody specifically binds to a cancer antigen.

Embodiment 2: The isolated antibody of embodiment 1, wherein the Kₒₙ is determined using surface plasmon resonance.

Embodiment 3: The antibody of embodiment 1, wherein the Kₒₙ is at least 10⁵ M⁻¹s⁻¹ or greater.

Embodiment 4: The antibody of embodiment 1, wherein said cancer antigen is CD20, HER2/neu, VEGF, EphA2, integrin αᵥβ3, EphA4, CD2, CD19, CD22, cMET, or ALK.

Embodiment 5: The antibody of embodiment 4, wherein said antibody specifically binds CD20 and exhibits a greater Kon when compared to rituximab.

Embodiment 6: The antibody of embodiment 4, wherein said antibody specifically binds HER2/neu and exhibits a greater Kon when compared to trastuzumab.

Embodiment 7: The antibody of embodiment 4, wherein said antibody specifically binds VEGF and exhibits a greater Kon when compared to bevacizumab.

Embodiment 8: The antibody of embodiment 1, wherein said antibody has an affinity constant (Ka) of at least about 10⁸ M⁻¹ or greater.

Embodiment 9: The antibody of embodiment 1, wherein said antibody has an affinity constant (Ka) of at least about 10⁹ M⁻¹ or greater.

Embodiment 10: The antibody of embodiment 1, wherein said antibody has an affinity constant (Ka) of at least about 10¹¹ M⁻¹ or greater.

Embodiment 11: The antibody of embodiment 1, wherein said antibody has an affinity constant (Ka) of at least about 10¹¹ M⁻¹ or greater.

Embodiment 12: The antibody of embodiment 1, wherein said antibody has an EC₅₀ of less than 6.0 nM.

Embodiment 13: The antibody of embodiment 1, wherein said antibody has an EC₅₀ of less than 3.0 nM.

Embodiment 14: The antibody of embodiment 1, wherein said antibody has an EC₅₀ of less than 1.0 nM.

Embodiment 15: A composition comprising the antibody of embodiment 1,wherein said composition does not exhibit a binding site barrier effect when administered to a solid tumor.

Embodiment 16: The composition of embodiment 15, wherein the composition demonstrates intra-tumoral diffusion, as measured by immunohistochemistry (IHC).

Embodiment 17: The composition of embodiment 16, wherein the intra-tumoral diffusion is greater than 3 tumor cell diameters.

Embodiment 18: The composition of embodiment 16, wherein the intra-tumoral diffusion is greater than 100 tumor cell diameters.

Embodiment 19: The composition of embodiment 15, wherein the composition demonstrates a specific tumor retention of at least 0.2% injected dose per gram of tumor (ID/g).

Embodiment 20: The composition of embodiment 19, wherein the specific tumor retention is at least 0.5%.

Embodiment 21: The composition of embodiment 19, wherein the specific tumor retention is at least 1.0%.

Embodiment 22: The composition of embodiment 19, wherein the specific tumor retention is at least 1.5%.

Embodiment 23: The antibody of embodiment 1, wherein said antibody is covalently linked to a cytotoxic moiety, such as a radionucleotides, toxins or chemotherapeutic agents.

Embodiment 24: The antibody of embodiment 1, wherein said antibody is a monoclonal antibody.

Embodiment 25: The antibody of embodiment 1, wherein said antibody is a Fab or F(ab′)2 fragment or a scFv or a single-chain antibody or a chimeric antibody.

Embodiment 26: The antibody of embodiment 1, wherein said antibody is a humanized antibody.

Embodiment 27: The antibody of embodiment 1, wherein said antibody is a human antibody.

Embodiment 28: The composition of embodiments 4, 5, 6, 7, 15, 17 or 19, comprising a pharmacologically acceptable diluent or excipient.

Embodiment 29: A composition comprising the antibody of embodiment 23, wherein said composition comprises a pharmacologically acceptable diluent or excipient.

Embodiment 30: The antibody of embodiment 1, wherein said antibody further has a K_{off} of less than 1×10⁻³s⁻¹.

Embodiment 31: The antibody of embodiment 1, wherein said antibody further has a K_{off} of less than 10⁻⁴s⁻¹.

Embodiment 32: A method of treating a tumor in a patient in need thereof, comprising administering a therapeutically effective amount of the composition of embodiment 15, 28 or 29.

### DEFINITIONS

An “isolated” or “purified” antibody is substantially free of cellular material or other contaminating proteins from the cell or tissue source from which the protein is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, or 5% (by dry weight) of heterologous protein (also referred to herein as a “containinating protein”). When the antibody is recombinantly produced, it is also preferably substantially free of culture medium, i.e., culture medium represents less than about 20%, 10%, or 5% of the volume of the protein preparation. When the antibody is produced by chemical synthesis, it is preferably substantially free of chemical precursors or other chemicals, i.e., it is separated from chemical precursors or other chemicals which are involved in the synthesis of the protein. Accordingly such preparations of the antibody have less than about 30%, 20%, 10%, 5% (by dry weight) of chemical precursors or compounds other than the antibody of interest. In a preferred embodiment, antibodies of the invention are isolated or purified.

As used herein, the terms “antibody” and “antibodies” refer to monoclonal antibodies, multispecific antibodies, human antibodies, humanized antibodies, chimeric antibodies, and anti-idiotypic (anti-Id) antibodies (including, e.g., anti-Id antibodies to antibodies of the invention). In particular, antibodies include immunoglobulin molecules i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

An “antibody fragment” refers to any fragment of the above described antibodies, including single-chain Fvs (scFv), single chain antibodies, Fab fragments, F(ab′) fragments, disulfide-linked Fvs (sdFv), camelised antibodies, and immunologically active fragments of immunoglobulin molecules, and epitope-binding fragments of any of the above.

An “agonistic” anti-tumor antibody refers to an antibody that can elicit signaling mechanisms in a cell e.g., increase the target antigen's phosphorylation and/or to degrade the target antigen. An agonistic antibody can prevent the onset or recurrence of cancer in patients predisposed to having cancer. As a non-limiting example, an agonistic antibody may be, for example, an agonistic anti-EphA2 antibody, as further defined herein.

An “antagonistic” anti-tumor antibody refers to an antibody that specifically targets a tumor antigen and results in a function-blocking of the cell signaling mechanism or other biological activity required for the tumor to progress. An antagonistic antibody can prevent the onset or recurrence of cancer in patients predisposed to having cancer. As a non-limiting example, an antagonistic antibody may be, for example, an anti-VEGF antibody, as further defined herein.

“EphA2” refers to a 130 kDa receptor tyrosine kinase that is expressed in adult epithelia, where it is found at low levels and is enriched within sites of cell-cell adhesion (Zantek et al., 1999, Cell Growth & Differentiation 10(9):629-38; Lindberg et al., Mol. & Cell. Biol. 10:6316, 1990). This subcellular localization is thought to play a role in contact inhibition through the interaction of EphA2 with its ligands (known as EphrinsA1 to A5) that are anchored to the cell membrane on adjacent cells (Eph Nomenclature Committee, 1997, Cell 90:403-04; Cheng et al., 2002, Cytokine & Growth Factor Rev. 13:75-85). Engagement of EphA2 with its ligand results in autophosphorylation of EphA2 and its subsequent degradation (Walker-Daniels et al., 2002, Mol. Cancer. Res. 1(1):79-87; Carles-Kinch et al., 2002, Cancer Res. 62(10):2840-47). This signaling cascade also initiates downstream events that negatively regulate attachment to extracellular matrix adhesions and thereby regulate cell growth and migration (Zantek et al., 1999, Cell Growth & Differentiation 10(9):629-38; Miao et al., 2000, Nat. Cell Biol. 2(2):62-69; Zelinski et al., 2001, Cancer Res. 61(5):230l -06).

EphA2 has been shown to be overexpressed in a number of different tumor types including melanoma, renal cell carcinoma, breast, prostate, colon, esophageal, cervical, lung, ovarian and bladder cancers (Carles-Kinch et al., 2002, Cancer Res. 62(10):2840-47). The highest levels of EphA2 expression are observed in the most aggressive cells, suggesting a role for EphA2 in disease progression. High levels of EphA2 have also been correlated with poor survival for non-small cell lung, esophageal, cervical and ovarian cancers (Kinch et al., 2003, Clin. Cancer Res. 9(2):613-18; Miyazaki et al., 2003, Int. J. Cancer 103(5) 657-63; Wu et al., 2004, Gynecol. Oncol. 94(2):312-19; Thaker et al., 2004, Clin. Cancer Res. 10(15):5145-50). Additionally, in pre-clinical models, it has been demonstrated that exogenous expression of EphA2 is sufficient to render a non-tumorigenic cell line tumorigenic in vitro and in vivo (Zelinski et al., 2001, Cancer Res. 61(5):2301-06).

The anti-EphA2 antibodies disclosed herein include: EA2, EA3, EA4, EA5, 3F2, 4H5, Eph099B-102.147, Eph099B-208.261, Eph099B-210.248, Eph099B-233.152, Eph101.530.241, 233 and G5. See, e.g., U.S. Patent Pub. Nos. US 2004-0091486 A1 (May 13, 2004), US 2004-0028685 A1 (Feb. 12, 2004), US 2005-0059592 A 1 (Mar. 17, 2005), US 2005-0048617 A1, U.S. application Ser. No. 11/165,023, filed Jun. 24, 2005 and Ser. No. 11/203,251, filed Aug. 15, 2005, each of which is incorporated by reference herein in its entirety. Hybridomas producing antibodies EA2 (strain EA2.31) and EA5 (strain EA5.12) of the invention have been deposited with the American Type Culture Collection (ATCC, P.O. Box 1549, Manassas, Va. 20108) on May 22, 2002, and assigned accession numbers PTA-4380 and PTA-4381, respectively and incorporated by reference. Eph099B-102.147, Eph099B-208.261, and Eph099B-210.248 were deposited with the ATCC on Aug. 7, 2002 and assigned accession nos. (PTA-4572, PTA-4573, and PTA-4574, respectively). Eph099B-233.152 was deposited with the ATCC on May 12, 2003 and assigned accession no. PTA-5194, and Eph101.530.241 was deposited with the ATCC on Sep. 26, 2002 and assigned accession no. PTA-4724. All of the aforementioned deposits were made under the provisions of the Budapest Treaty on the International Recognition of the Deposit of Microorganisms for the Purposes of Patent Procedures, and the accession numbers and dates corresponding to the respective antibodies are incorporated by reference herein in their entireties.

As used herein, an “improved recombinant anti-tumor antibody” refers to an antibody of the invention which specifically binds a particular tumor or cancer antigen as defined herein, and which has been engineered or screened for certain desirable kinetic properties, such as, for example, a increased or faster Kon rate, as compared to the same recombinant anti-tumor antibody without such engineered or screened for changes. In addition, such an improved recombinant anti-tumor antibody may also be engineered or screened for an increased or faster Koff rate as compared with the same without such engineered or screened for changes.

As used herein, “intra-tumoral diffusion” is the tumor cell diameter distance traveled by an anti-tumor antibody of the invention that specifically targets said tumor cell(s). Intra-tumoral diffusion can be greater than 3, 5, 10, 20, 30, 40, 50, 60, 70, 80, 90 or 100 tumor cell diameters as measured by immunohistochemistry (IHC) and is useful as a measure of tumor penetrance of a particular improved anti-tumor antibody, particularly when compared to the same anti-tumor antibody before such improvement.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “patchy staining” or “patchy tumor penetrance” refers to the visual or observed quantitation of immunohistochemical staining of tumor cells/tissues by a particular anti-tumor antibody of the invention, typically one that is not improved. A patchy tumor penetrance or staining indicates a less desirable tumor penetration of a particular anti-tumor antibody that is not improved. Such may be quantified by an estimated percent of visualized tumor staining, as measured by IHC.

As used herein, the term “patient” is preferably a mammal such as a non-primate (e.g., cows, pigs, horses, cats, dogs, rats, etc.) and a primate (e.g., monkey (e.g., a rhesus monkey, a cynomolgus monkey or chimpanzee) and human, most preferably a human. In one embodiment, the patient is a mammal, preferably a human, with a disorder associated with expression and/or activity of a tumor. Such disorders include, but are not limited to, cancer, non-cancer hyperproliferative cell disorders, and infections. In another embodiment, the patient is a mammal, preferably a human, at risk of developing a disorder associated with expression and/or activity of a tumor (e.g., an immunocompromised or immunosuppressed mammal, or a genetically predisposed mammal). In another embodiment, the subject is not an immunocompromised or immunosuppressed mammal, preferably a human. In another embodiment, the patient is a mammal, preferably a human, with a lymphocyte count that is not under approximately 500 cells/mm³.

As used herein, the terms “treat”, “treatment” and “treating in the context of administering a therapy(ies) to a patient refer to the reduction or amelioration of the progression, severity, and/or duration of a disorder associated with aberrant expression (e.g., overexpression) of a tumor antigen or activity of a cancer, a non-cancer hyperproliferative cell disorder or an infection, and/or the amelioration of one or more symptoms thereof resulting from the administration of one or more therapies (including, but not limited to, the administration of one or more prophylactic or therapeutic agents).

As used herein, the term “uniform staining” or “uniform tumor penetrance” refers to the visual or observed quantitation of immunohistochemical staining of tumor cells/tissues by a particular improved anti-tumor antibody of the invention. A uniform tumor penetrance or staining indicates a desirable tumor penetration of the particular improved anti-tumor antibody of the invention. Such may be quantified by an estimated percent of visualized tumor staining, as measured by IHC. Such IHC measurements may be quantitated as having at least 70% IHC staining, or at least 80% IHC staining, or at least 90% IHC staining or 100% IHC staining.

### DESCRIPTION OF FIGURES

FIG. 1: Immunohistochemical (IHC) staining of a negative control antibody R347 which does not bind human EphA2 tumor antigen. See Table 2.

FIGS. 2A-H: IHC staining of G5 anti-EphA2 antibody, considered the reference antibody against which the other tested anti-EphA2 antibodies were judged. The panels A-H represent the time course of tumor harvest wherein A=30 min, B=1 hr, C=2 hrs, D=8 hrs, E=16 hrs, F=24 hrs, G=36 hrs, and H=48 hrs. See Table 2.

FIGS. 3A-H: IHC staining of 3F2 anti-EphA2 antibody. The panels A-H represent the time course of tumor harvest wherein A=30 min, B=1 hr, C=2 hrs, D=8 hrs, E=16 hrs, F=24 hrs, G=36 hrs, and H=48 hrs. See Table 2.

FIGS. 4A-H: IHC staining of 7A9 anti-EphA2 antibody. The panels A-H represent the time course of tumor harvest wherein A=30 min, B=1 hr, C=2 hrs, D=8 hrs, E=16 hrs, F=24 hrs, G=36 hrs, and H=48 hrs. See Table 2.

FIGS. 5A-H: IHC staining of 4H5 shuffled anti-EphA2 antibody. The panels A-H represent the time course of tumor harvest wherein A=30 min, B=1 hr, C=2 hrs, D=8 hrs, E=16 hrs, F=24 hrs, G=36 hrs, and H=48 hrs. See Table 2.

FIGS. 6A-H: IHC staining of 3D10 anti-EphA2 antibody. The panels A-H represent the time course of tumor harvest wherein A=30 min, B=1 hr, C=2 hrs, D=8 hrs, E=16 hrs, F=24 hrs, G=36 hrs, and H=48 hrs. See Table 2.

FIGS. 7A-H: IHC staining of 4H5 corrected anti-EphA2 antibody. The panels A-H represent the time course of tumor harvest wherein A=30 min, B=1 hr, C=2 hrs, D=8 hrs, E=16 hrs, F=24 hrs, G=36 hrs, and H=48 hrs. See Table 2.

### DETAILED DESCRIPTION OF THE INVENTION

In one embodiment, the improved anti-tumor antibodies of the invention specifically bind to its tumor target with a Kon of at least 10⁴ M⁻¹s⁻¹ or greater, at least 10⁵ M⁻¹s⁻¹ or greater, at least 5×10⁵ M⁻¹s⁻¹ or greater, at least 10⁶ M⁻¹s⁻¹ or greater, at least 5×10⁶ M⁻¹s⁻¹ or greater, at least 10⁷ M⁻¹s⁻¹ or greater, at least 5×10⁷ M⁻¹s⁻¹ or greater, at least 10⁸ M⁻¹s⁻¹ or greater, or at least 10⁹ M⁻¹s⁻¹ or greater as measured by surface plasmon resonance (SPR).

In another embodiment, the improved anti-tumor antibodies of the invention specifically bind to its tumor target with a Kon rate that is at least 2-fold greater than the same antibody without said Kon improvement, at least 3-fold greater than the same antibody without said Kon improvement, at least 4-fold greater than the same antibody without said Kon improvement, at least 5-fold greater than the same antibody without said Kon improvement, at least 6-fold greater than the same antibody without said Kon improvement, at least 7-fold greater than the same antibody without said Kon improvement, at least 8-fold greater than the same antibody without said Kon improvement, at least 9-fold greater than the same antibody without said Kon improvement, at least 10-fold greater than the same antibody without said Kon improvement, at least 11-fold greater than the same antibody without said Kon improvement, at least 12-fold greater than the same antibody without said Kon improvement, at least 13-fold greater than the same antibody without said Kon improvement, at least 14-fold greater than the same antibody without said Kon improvement, at least 15-fold greater than the same antibody without said Kon improvement, at least 16-fold greater than the same antibody without said Kon improvement, at least 17-fold greater than the same antibody without said Kon improvement, at least 18-fold greater than the same antibody without said Kon improvement, at least 19-fold greater than the same antibody without said Kon improvement, at least 20-fold greater than the same antibody without said Kon improvement, at least 21-fold greater than the same antibody without said Kon improvement, at least 22-fold greater than the same antibody without said Kon improvement, at least 23-fold greater than the same antibody without said Kon improvement, at least 24-fold greater than the same antibody without said Kon improvement, at least 25-fold greater than the same antibody without said Kon improvement, at least 26-fold greater than the same antibody without said Kon improvement, at least 27-fold greater than the same antibody without said Kon improvement, at least 28-fold greater than the same antibody without said Kon improvement, at least 29-fold greater than the same antibody without said Kon improvement, at least 30-fold greater than the same antibody without said Kon improvement, and so on, such that the improved anti-tumor antibody specifically binds its tumor antigen target at a faster association rate than the same antibody without such an improvement, as measured by surface plasmon resonance (SPR).

In another embodiment, the antibodies of the invention have an affinity constant or Kₐ (k(ₒₙ/k_{off}) of from about 10² M⁻¹ to about 5×10¹⁵ M⁻¹, preferably at least 10⁴ M⁻¹. In some embodiments, the antibody has a Kₐ of about 10⁸ M⁻¹, of about 10⁹ M⁻¹, of about 10¹⁰ M⁻¹, and of about 10¹¹ M⁻¹, as measured by surface plasmon resonance (SPR).

In another embodiment, the improved anti-tumor antibodies of the invention specifically bind to its tumor target with a Koff of 1×10⁻³ s⁻¹ or less, or 3×10⁻³ s⁻¹ or less, 5×10⁻³ s⁻¹ or less, 10⁻⁴ s⁻¹ or less, 5×10⁻⁴ s⁻¹ or less, 10⁻⁵ s⁻¹ or less, 10⁻⁶ s⁻¹ or less, 5×10⁻⁶ s⁻¹ or less, 10⁻⁷ s⁻¹ or less, 5×10⁻⁷ s⁻¹ or less, 10⁻⁸ s⁻¹ or less, 5×10⁻⁸ s⁻¹ or less, 10⁻⁹ s⁻¹ or less, 5×10⁻⁹ s⁻¹ or less, or 10⁻¹⁰ s⁻¹ or less, as measured by surface plasmon resonance (SPR).

In yet another embodiment, the improved anti-tumor antibodies of the invention specifically bind to its tumor target with a Koff rate of at least 2-fold less than the same antibody without said Koff improvement, at least 3-fold less than the same antibody without said Koff improvement, at least 4-fold less than the same antibody without said Koff improvement, at least 5-fold less than the same antibody without said Koff improvement, at least 6-fold less than the same antibody without said Koff improvement, at least 7-fold less than the same antibody without said Koff improvement, at least 8-fold less than the same antibody without said Koff improvement, at least 9-fold less than the same antibody without said Koff improvement, at least 10-fold less than the same antibody without said Koff improvement, at least 11-fold less than the same antibody without said Koff improvement, at least 12-fold less than the same, antibody without said Koff improvement, at least 13-fold less than the same antibody without said Koff improvement, at least 14-fold less than the same antibody without said Koff improvement, at least 15-fold less than the same antibody without said Koff improvement, at least 16-fold less than the same antibody without said Koff improvement, at least 17-fold less than the same antibody without said Koff improvement, at least 18-fold less than the same antibody without said Koff improvement, at least 19-fold less than the same antibody without said Koff improvement, at least 20-fold less than the same antibody without said Koff improvement, at least 21-fold less than the same antibody without said Koff improvement, at least 22-fold less than the same antibody without said Koff improvement, at least 23-fold less than the same antibody without said Koff improvement, at least 24-fold less than the same antibody without said Koff improvement, at least 25-fold less than the same antibody without said Koff improvement, at least 26-fold less than the same antibody without said Koff improvement, at least 27-fold less than the same antibody without said Koff improvement, at least 28-fold less than the same antibody without said Koff improvement, at least 29-fold less than the same antibody without said Koff improvement, at least 2-fold less than the same antibody without said Koff improvement, and so on, such that the improved anti-tumor antibody dissociates from its specific tumor antigen target in a faster manner as compared to the same antibody without said Koff improvement, as measured by surface plasmon resonance (SPR).

In another embodiment, the improved anti-tumor antibodies of the invention specifically bind to its tumor target and have a dissociation constant (K_{d}) of less than 10000 pM, less than 3000 pM, less than 2500 pM, less than 2000 pM, less than 1500 pM, less than 1000 pM, less than 750 pM, less than 500 pM, less than 250 pM, less than 200 pM, less than 150 pM, less than 100 pM, less than 75 pM as assessed using an described herein or known to one of skill in the art (e.g., a BIAcore assay). In another embodiment, the improved anti-tumor antibodies of the invention specifically bind to its tumor target and have a dissociation constant (K_{d}) of between 25 to 10000 pM, 25 to 3400 pM, 25 to 3000 pM, 25 to 2500 pM, 25 to 2000 pM, 25 to 1500 pM, 25 to 1000 pM, 25 to 750 pM, 25 to 500 pM, 25 to 250 pM, 25 to 100 pM, 25 to 75 pM, 25 to 50 pM as assessed using an described herein or known to one of skill in the art (e.g., a BIAcore assay).

The antibody kinetics described and claimed in the present invention can be measured by surface plasmon resonance (SPR) measurement using, for example, a BIAcore 2000 (BIAcore Inc.) as described previously (Popov et al., Mol. Immunol., 33:493-502, 1996; Karlsson et al., J. Immunol. Methods, 145:229-240, 1991, both of which are incorporated by reference in their entireties). In this method, antibody molecules are coupled to a BIAcore sensor chip (e.g., CM5 chip by Pharmacia) and the binding of the antibodies of the invention to the immobilized target is measured at a certain flow rate to obtain sensorgrams using BIA evaluation 2.1 software, based on which on- and off-rates of the antibodies of the invention, or fragments thereof, to the immobilized target can be calculated. Such calculations and methods of measurement are well within the skill of the ordinary artisan.

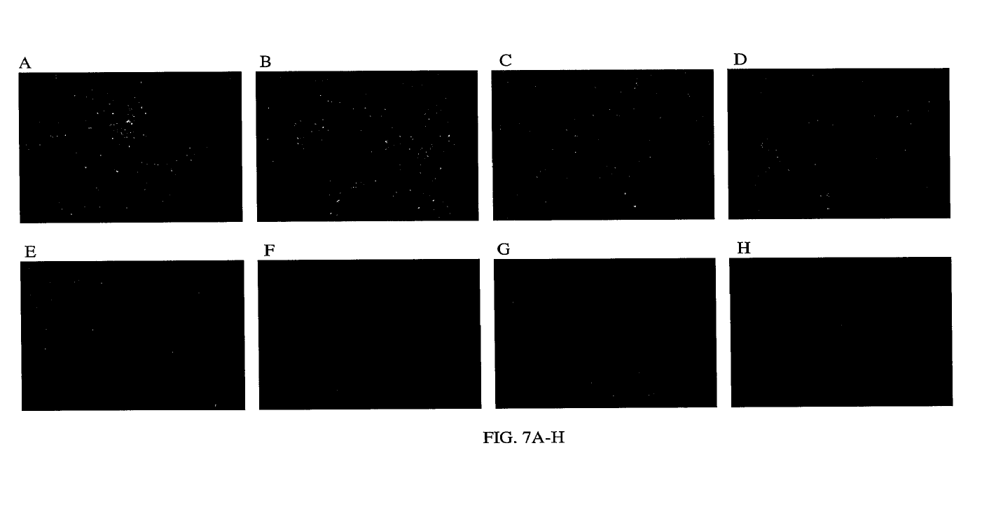

The present invention provides for improved antibodies that exhibit a high potency in an assay described herein. High potency antibodies can be produced by methods disclosed in copending U.S. patent application Ser. Nos. 60/168,426, 60/186,252, U.S. Publication No. 2002/0098189, and U.S. Pat. No. 6,656,467 (which are incorporated herein by reference in their entirety), as well as in Wu, H. et. al., J. Mol. Biol. (2005) 350:126-144, particularly the described Kon optimization and novel ELISA screen section and FIG. 7, described therein. For example, high potency antibodies can be produced by genetically engineering appropriate antibody gene sequences and expressing the antibody sequences in a suitable host. The antibodies produced can be screened to identify antibodies with, e.g., high kₒₙ values in a BIAcore assay or alternatively, in a sensitive ELISA screen (see Wu, H. et. al., J. Mol. Biol. (2005) 350:126-144).

The effector functions of an improved antibody of the invention can be measured by an ADCC assay. Chromium assays are well-known in the art (see, for example, Brunner, K. T. et al., (1968) Quantitative Assay of the Lytic Action of Immune Lymphoid Cells on Cr-labelled Allogenic Target Cells in-vitro; Inhibition by Iso-antibody and by Drugs, Immunology 14,181). More recently, LDH cytotoxicity assays are being used. The assay is based on measurement of activity of lactate dehydrogenase (LDH) which is a stable enzyme normally found in the cytosol of all cells but rapidly releases into the supernatant upon damage of plasma membrane. Results can be analyzed by spectrophotometry at 500 nm. Such assays are available commercially as kits, therefore are readily available to those of skill in the art.

Improved antibodies of the invention that immunospecifically bind to a tumor antigen can be produced by any method known in the art for the synthesis of antibodies, in particular, by chemical synthesis or preferably, by recombinant expression techniques. The practice of the invention employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press. Further, the antibodies of the present invention can also be generated using various phage display methods known in the art.

For example, antibodies can also be generated using various phage display methods. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/O1 134; International Publication Nos. WO 90/02809, WO 91/10737, WO 92/01047, WO 92/18619, WO 93/1 1236, WO 95/15982, WO 95/20401, and W097/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)₂ fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415, which are incorporated herein by reference in their entirety.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains (Fab, Fab′, F(ab′)₂, Fabc, Fv) in which all or substantially all of the CDR regions correspond to those of a non human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG1. Where such cytotoxic activity is not desirable, the constant domain may be of the IgG2 class. Examples of VL and VH constant domains that can be used in certain embodiments of the invention include, but are not limited to, C-kappa and C-gamma-1 (nG1m) described in Johnson et al. (1997) J. Inject. Dis. 176, 1215-1224 and those described in U.S. Pat. No. 5,824,307. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental FR and CDR sequences, more often 90%, and most preferably greater than 95%. Humanized antibodies can be produced using variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), which is incorporated by reference herein in its entirety. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Reichmann et al., 1988, Nature 332:323, which are incorporated herein by reference in their entireties).

The invention provides polynucleotides comprising a nucleotide sequence encoding an improved antibody (modified) of the invention that immunospecifically binds to a particular tumor antigen of interest (e.g., EphA2 antigen).

The polynucleotides may be obtained, and the nucleotide sequence of the polynucleotides determined, by any method known in the art i.e., nucleotide codons known to encode particular amino acids are assembled in such a way to generate a nucleic acid that encodes the antibody. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, fragments, or variants thereof, annealing and ligating of those oligonucleotides, and then amplification of the ligated oligonucleotides by PCR.

Alternatively, a polynucleotide encoding an improved antibody of the invention may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions. In certain embodiments, amino acid substitutions, deletions and/or insertions are introduced into the epitope-binding domain regions of the antibodies and/or into the hinge-Fc regions of the antibodies which are involved in the interaction with the FcRn.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278:457-479 for a listing of human framework regions). Preferably, the polynucleotide sequence generated by the combination of the framework regions and CDRs encodes an antibody that immunospecifically binds to a particular tumor antigen. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

Mutagenesis may be performed in accordance with any of the techniques known in the art including, but not limited to, synthesizing an oligonucleotide having one or more modifications within the sequence of the constant domain of an antibody or a fragment thereof (e.g., the CH2 or CH3 domain) to be modified. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered. A number of such primers introducing a variety of different mutations at one or more positions may be used to generated a library of mutants.

The technique of site-specific mutagenesis is well known in the art, as exemplified by various publications (see, e.g.,. Kunkel et al., Methods Enzymol., 154:367-82, 1987, which is hereby incorporated by reference in its entirety). In general, site-directed mutagenesis is performed by first obtaining a single-stranded vector or melting apart of two strands of a double stranded vector which includes within its sequence a DNA sequence which encodes the desired peptide. An oligonucleotide primer bearing the desired mutated sequence is prepared, generally synthetically. This primer is then annealed with the single-stranded vector, and subjected to DNA polymerizing enzymes such as T7 DNA polymerase, in order to complete the synthesis of the mutation-bearing strand. Thus, a heteroduplex is formed wherein one strand encodes the original non-mutated sequence and the second strand bears the desired mutation. This heteroduplex vector is then used to transform or transfect appropriate cells, such as E. coli cells, and clones are selected which include recombinant vectors bearing the mutated sequence arrangement. As will be appreciated, the technique typically employs a phage vector which exists in both a single stranded and double stranded form. Typical vectors useful in site-directed mutagenesis include vectors such as the M13 phage. These phage are readily commercially available and their use is generally well known to those skilled in the art. Double stranded plasmids are also routinely employed in site directed mutagenesis which eliminates the step of transferring the gene of interest from a plasmid to a phage.

Alternatively, the use of PCR™ with commercially available thermostable enzymes such as Taq DNA polymerase may be used to incorporate a mutagenic oligonucleotide primer into an amplified DNA fragment that can then be cloned into an appropriate cloning or expression vector. See, e.g., Tomic et al., Nucleic Acids Res., 18(6):1656, 1987, and Upender et al., Biotechniques, 18(1):29-30, 32, 1995, for PCR™-mediated mutagenesis procedures, which are hereby incorporated in their entireties. PCR™ employing a thermostable ligase in addition to a thermostable polymerase may also be used to incorporate a phosphorylated mutagenic oligonucleotide into an amplified DNA fragment that may then be cloned into an appropriate cloning or expression vector (see e.g., Michael, Biotechniques, 16(3):410-2, 1994, which is hereby incorporated by reference in its entirety).

Other methods known to those of skill in art of producing sequence variants of the Fc domain of an antibody or a fragment thereof can be used. For example, recombinant vectors encoding the amino acid sequence of the constant domain of an antibody or a fragment thereof may be treated with mutagenic agents, such as hydroxylamine, to obtain sequence variants.

Vectors, in particular, phage, expressing constant domains or fragments thereof having one or more modifications in amino acid residues can be screened to identify constant domains or fragments thereof having increased or decreased affinity for FcRn. Immunoassays which can be used to analyze binding of the constant domain or fragment thereof having one or more modifications in amino acid residues to the FcRn include, but are not limited to, radioimmunoassays, ELISA (enzyme linked immunosorbent assay), “sandwich” immunoassays, and fluorescent immunoassays. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety). Exemplary immunoassays are described briefly herein below (but are not intended by way of limitation). BIAcore kinetic analysis can also be used to determine the binding on and off rates of a constant domain or a fragment thereof having one or more modifications in amino acid residues to the FcRn. BIAcore kinetic analysis comprises analyzing the binding and dissociation of a constant domain or a fragment thereof having one or more modifications in amino acid residues from chips with immobilized FcRn on their surface.

Any of a variety of sequencing reactions known in the art can be used to directly sequence the nucleotide sequence encoding, e.g., variable regions and/or constant domains or fragments thereof having one or more amino acid Fc domain modifications. Examples of sequencing reactions include those based on techniques developed by Maxim and Gilbert (Proc. Natl. Acad. Sci. USA, 74:560, 1977) or Sanger (Proc. Natl. Acad. Sci. USA, 74:5463, 1977). It is also contemplated that any of a variety of automated sequencing procedures can be utilized (Bio/Techniques, 19:448, 1995), including sequencing by mass spectrometry (see, e.g., PCT Publication No. WO 94/16101, Cohen et al., Adv. Chromatogr., 36:127-162, 1996, and Griffin et al., Appl. Biochem. Biotechnol., 38:147-159, 1993).

Recombinant expression of an antibody of the invention (e.g., a heavy or light chain of an antibody of the invention or a single chain antibody of the invention) that immunospecifically binds to a tumor antigen requires construction of an expression vector containing a polynucleotide that encodes the antibody. Once a polynucleotide encoding an antibody molecule, heavy or light chain of an antibody, or fragment thereof (preferably, but not necessarily, containing the heavy and/or light chain variable domain) of the invention has been obtained, the vector for the production of the antibody molecule may be produced by recombinant DNA technology using techniques well-known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a fragment thereof, or a heavy or light chain CDR, operably linked to a promoter. Such vectors may include the nucleotide sequence encoding the constant region of the antibody molecule (see, e.g., international Publication Nos. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or fragment thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies of the invention which immunospecifically bind to a tumor antigen is regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa califbrnica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NSO (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2197-2199). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention may be fused to heterologous polypeptide sequences described herein or otherwise known in the art to facilitate purification.

The present invention further encompasses uses of antibodies or fragments thereof conjugated to a therapeutic moiety. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Therapeutic moieties include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference), hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)), cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, procaine, tetracaine, lidocaine, propranolol, and puromycin and analogs or homologs thereof) and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459), farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305), topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN-1518B; KT6006; KT6528; ED-110; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate) HMG-CoA reductase inhibitors, (e.g., lovastatin, simvastatin, atorvastatin, pravastatin, fluvastatin, statin, cerivastatin, lescol, lupitor, rosuvastatin and atorvastatin) and pharmaceutically acceptable salts, solvates, clathrates, and prodrugs thereof. See, e.g., Rothenberg, M. L., Annals of Oncology 8:837-855(1997); and Moreau, P., et al., J. Med. Chem. 41:1631-1640(1998)), antisense oligonucleotides (e.g., those disclosed in the U.S. Pat. Nos. 6,277,832, 5,998,596, 5,885,834, 5,734,033, and 5,618,709), immunomodulators (e.g., antibodies and cytokines), antibodies, and adenosine deaminase inhibitors (e.g., Fludarabine phosphate and 2-Chlorodeoxyadenosine). Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), Auristatin molecules (e.g., auristatin PHE, bryostatin 1, solastatin 10, see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference), anti-mitotic agents (e.g., vincristine and vinblastine), hormones (e.g., glucocorticoids, progestatins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)), and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459), farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305), topoisomerase inhibitors (e.g., camptothecin; irinotecan; SN-38; topotecan; 9-aminocamptothecin; GG-211 (GI 147211); DX-8951f; IST-622; rubitecan; pyrazoloacridine; XR-5000; saintopin; UCE6; UCE1022; TAN-1518A; TAN-1518B; KT6006; KT6528; ED-110; NB-506; ED-110; NB-506; and rebeccamycin; bulgarein; DNA minor groove binders such as Hoescht dye 33342 and Hoechst dye 33258; nitidine; fagaronine; epiberberine; coralyne; beta-lapachone; BC-4-1; and pharmaceutically acceptable salts, solvates, clathrates, and prodrugs thereof. See, e.g., Rothenberg, M. L., Annals of Oncology 8:837-855(1997); and Moreau, P., et al., J. Med. Chem. 41:1631-1640(1998)), antisense oligonucleotides (e.g., those disclosed in the U.S. Pat. Nos. 6,277,832, 5,998,596, 5,885,834, 5,734,033, and 5,618,709), immunomodulators (e.g., antibodies and cytokines), antibodies (e.g., rituximab (Rituxan®), calicheamycin (Mylotarg®), ibritumomab tiuxetan (Zevalin®), and tositumomab (Bexxar®)), and adnosine deaminase inhibitors (e.g., Fludarabine phosphate and 2-Chlorodeoxyadenosine).

Non-limiting examples of chemotherapeutic agents include methotrexate, cyclosporin A, leflunomide, cisplatin, ifosfamide, taxanes such as taxol and paclitaxol, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, cytochalasin B, gramicidin D, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin homologs, and cytoxan.

Moreover, an antibody of the invention can be conjugated to therapeutic moieties such as a radioactive metal ion, such as alph-emiters such as ²¹³Bi or macrocyclic chelators useful for conjugating radiometal ions, including but not limited to, ¹³¹ln, ¹³¹LU, ¹³¹Y, ¹³¹Ho, ¹³¹Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties. Other radioisotopes may be 1-125, palladium, and iridium or strontium-89.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The methods and compositions of the invention are useful in preventing, managing, treating or ameliorating cancers and/or solid tumors. Specific examples of cancers that can be prevented, managed, treated or ameliorated in accordance with the invention include, but are not limited to, solid tumors of the head, neck, eye, mouth, throat, esophagus, chest, bone, lung, colon, rectum or other gastrointestinal tract organs, stomach, spleen, skeletal muscle, subcutaneous tissue, prostate, breast, ovaries, testicles or other reproductive organs, skin, thyroid, blood, lymph nodes, kidney, liver, pancreas, and brain or central nervous system.

Cancers and related disorders that can be treated or prevented by methods and compositions of the present invention include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypernephroma, fibrosarcoma, transitional cell cancer (renal pelvis and/ or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Accordingly, the methods and compositions of the invention are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the skin, lung, colon, breast, prostate, bladder, kidney, pancreas, ovary, or uterus. In other specific embodiments, sarcoma, melanoma, or leukemia is treated or prevented.

In some embodiments, the tumor is overexpresses a specific tumor antigen, such as, for example, CD20, HER2/neu, VEGF, EphA2, integrin αᵥβ3, EphA4, CD2, CD19, CD22, cMET, ALK, DLL4, or CEA. In other embodiments, the disorder to be treated is a pre-cancerous condition associated with cells that overexpress said tumor antigens. In a specific embodiments, the pre-cancerous condition is high-grade prostatic intraepithelial neoplasia (PIN), fibroadenoma of the breast, fibrocystic disease, or compound nevi.

In particular embodiments, the antibodies of the invention have in vivo therapeutic and/or prophylactic uses. Examples of therapeutic and prophylactic antibodies which may be so modified include, but are not limited to, HERCEPTIN® (Trastuzumab) (Genentech, CA) which is a humanized anti-HER2 monoclonal antibody for the treatment of patients with metastatic breast cancer; AVASTIN® (bevacizumab) humanized anti-CD18 F(ab′)₂ (Genentech, CA); CDP860 which is a humanized anti-CD 18 F(ab′)₂ (Celltech, UK); MAK-195 (SEGARD) which is a murine anti-TNF-α F(ab′)₂ (Knoll Phanna/BASF); IC14 which is an anti-CD14 antibody (ICOS Pharm); a humanized anti-VEGF IgG1 antibody (Genentech); OVAREX™ which is a murine anti-CA 125 antibody (Altarex); PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); ABEGRIN™ (etaracizumab) otherwise known as VITAXIN® which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN® (rituximab) which is a chimeric anti-CD20 IgG1 antibody (IDEC Pharm/Genentech, Roche/Zettyaku); LYMPHOCIDE™ which is a humanized anti-CD22 IgG antibody (Immunomedics); Smart ID10 which is a humanized anti-HLA antibody (Protein Design Lab); ABX-IL8 is a human anti-IL8 antibody (Abgenix); anti-CD 11a is a humanized IgG1 antibody (Genetech/Xoma); ICM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celitech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); MDX-33 is a human anti-CD64 (FcγR) antibody (Medarex/Centeon); SCH55700 is a humanized anti-IL-5 IgG4 antibody (Celltech/Schering); SB-240563 and SB-240683 are humanized anti-IL-5 and IL-4 antibodies, respectively, (SmithKline Beecham); IDEC-152 is a primatized anti-CD23 antibody (IDEC Pharm); ABX-CBL is a murine anti CD-147 IgM antibody (Abgenix); BTI-322 is a rat anti-CD2 IgG antibody (Medimmune/Bio Transplant); Orthoclone/OKT3 is a murine anti-CD3 IgG2a antibody (ortho Biotech); SIMULECT™ is a chimeric anti-CD25 IgG1 antibody (Novartis Pharm); LDP-01 is a humanized anti-13₂-integrin IgG antibody (LeukoSite); Anti-LFA-1 is a murine anti CD18 F(ab′)₂ (Pasteur-Merieux/Immunotech); anti-CD2 humanized antibody (MEDI-507) (Medlmmune, Inc., MD); anti-EphA2 humanized antibody (MEDI-531) (Medlmmune, Inc., MD); anti-EphA4 humanized antibody (MEDI-584) (Medlmmune, Inc., MD); anti-CD19 antibody; anti-CD22 antibody; anti-cMET antibody; or an anti-ALK antibody, or an anti-DLL4 antibody, or an anti-CEA antibody.

The present invention also provides compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

The amount of the therapeutic or compound used in the method of the invention is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the therapeutic or compound used in the method of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides; isopropyl amine, triethylamine, 2-ethylamino ethanol, histidine, procaine, etc.

The amount of the therapeutic or compound used in the method of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

Various delivery systems are known and can be used to administer a therapeutic in the method of the invention, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradennal, intramuscular, intraperitoneal, intravenous, subcutaneous, intranasal, epidural, and oral routes. The therapeutics or compounds described herein may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In yet another embodiment, the therapeutic or compound can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Press, Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al.,1989, J. Neurosurg. 71:105). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (1990, Science 249:1527-1533).

Accordingly, therapeutic compounds used in the method of the invention, designed for oral, lingual, sublingual, buccal and intrabuccal administration can be made without undue experimentation by means well known in the art, for example, with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and non-toxic in the amounts used.

### Examples

The invention is now described with reference to the following examples. These examples are provided for the purpose of illustration only and the invention should in no way be construed as being limited to these examples but rather should be construed to encompass any and all variations which become evident as a result of the teachings provided herein.

### Example 1

### Cloning of Parental Antibody into Phage Vector

The VL and VH genes of the parental antibody can be cloned into one M 13-based phage vector under the control of the lacZ promoter by hybridization mutagenesis, methods well-known to those of skill in the art (see Kunkel T. A. PNAS 82, 488-492 (1985), herein incorporated by reference). The vector will contain the backbone of the human kappa constant region (Cκ), the first constant region of the human γ1 chain (CH1), and two annealing sites for the cloning of VL and VH genes. The vector should also contain a pel B leader sequence for the light chain and a pho A leader sequence for the heavy chain to target the expressed Fab fragments into periplasmic space in E. Coli.

Briefly, the forward primers for both VL and VH genes can be biotinylated to facilitate the purification of minus-strand V genes at the later step for annealing into phage vector. The forward primers contain sequence specific to the framework 1 region and an over-hanging sequence annealed to the end of the leader sequence (pel B or pho A). Similarly, the reverse primers can contain sequence specific to the framework 4 region and an overhanging sequence annealed to the beginning of the constant region Cκ or C_{H}1.

The minus single-stranded DNA can be isolated by the dissociation of the double-stranded PCR product with sodium hydroxide while the plus biotinylated strand is captured by strepavidin-coated magnetic beads. A uridinylated template is prepared by any method known to one of skill in the art (see Wu, H. and An, L. Methods in Molecular Biology 207:213-233 (2003), herein incorporated by reference). Next, hybridization mutagenesis is performed and confirmed by DNA sequencing (see id).

### Example 2

### Construction of Focused CDR Libraries

For CDR library construction, the parental CDR region needs to be deleted first to avoid the domination of the library by the parental clone. In a successful single-site hybridization mutagenesis, the mutagenesis rate is usually between 50-80%. If the parental antibody is used as a template for the library construction, there will be 20 to 50% of the library population that is parent, and this will increase the difficulty of screening. The CDR region can be deleted by mutagenesis (see id). The oligonucleotide for mutagenesis is designed to replace the CDR region with a stop codon (TAA) and an extra nucleotide (A) to cause a frameshift. After the ΔCDR is made, it is used as a template for the construction of its corresponding CDR library. Altogether six CDR-deleted templates can be made corresponding to individual CDR libraries. To construct focused CDR libraries, the codon-based mutagenesis approach can be used to synthesize the oligonucleotides coding for CDR mutations (see Glaser S. M., et al., J. Immunol. 149, 3903-3913 (1992), herein incorporated by reference). With modification, one can synthesize the oligonucleotides containing single, double, triple mutations, etc. The oligonucleotides are then used for library construction by hybridization mutagenesis. For affinity maturation, single-mutation libraries can be sufficient. If significant characteristic change is desired for the antibody, double and more mutations may be needed.

### Example 3

### Screening of Focused CDR Libraries

The screening strategy can affect what kind of antibody variants are selected from the library. One can mimic the conditions used in the kinetic simulation to select the higher affinity variants either driven by Koff or by Kon. First, a capturing reagent is used to capture a constant amount of antibody variants for the assay. Antigen concentration should be lower than the Kd value to favor high affinity clones.

To screen a library exhaustively with high confidence, three times the size of the library is screened. Typical mutagenesis rates for library construction are ≧50%, thus a twofold factor needs to be adjusted for the mutagenesis background. Since each single-mutation CDR library normally contains less than 400 variants, approximately 2400 clones (=400×3×2) need to be screened per library.

### Example 3.A

### Koff Driven Screening

For Koff driven screening, improved variants are isolated under conditions allowing interactions between antibody and antigen for at least 1 hour; then the dissociation is carried out by frequent wash and incubation with wash buffer.

Two assays can be used for Koff—driven screening. One is the capture lift assay (see Watkins, J. D. et al., Anal. Biochem. 256, 169-177 (1998), herein incorporated by reference) and the other is the single point ELISA assay (SPE) (see Watkins, J. D., et al., Anal. Biochem. 253, 37-45 (1997), herein incorporated by reference). Confirm the Koff variants by ELISA titration on immobilized antigen (see Wu, H. and An, L. Methods in Molecular Biology 207:213-233 (2003), herein incorporated by reference).

### Example 3.B

### Kon Driven Screening by SPE

Developing a reliable assay to screen for the variants with improved Kon values is more difficult than developing an assay to screen Koff-based improved clones. In the current example, the ELISA assay, Kon SPE is established as described in Wu, H. and An, L. Methods in Molecular Biology 207:213-233 (2003), herein incorporated by reference.

To select for variants with improved Kon value, both the association and dissociation time need to be as short as possible (preferably ≦10 minutes total). Usually this process will result in a weak signal output. A signal amplification step may be added to increase the signal to noise ratio, using any readily available technique well-known to the skilled artisan.

First, coat Immunlon 1B plate with goat anti-human kappa antibody at 1 ug/mL in carbonate coating buffer at 4° C. overnight. Tap out the coating solution. Block with 1% BSA/PBS (200 uL/well) for 1 hour at RT. Remove the BSA, and add 200 uL of Fab extract from periplasmic preparation to each well. Incubate for 2 hrs at RT. Wash 3× with PBS/0.1% Tween-20. Add 50 uL/well of antigen-horseradish peroxidase (HRP) complex, and incubate for 10 minutes at RT. Wash 3× with PBS/0.1% Tween-20 quickly. Add TMB substrate, and incubate for 15 minutes at RT. Stop the reaction with 0.5 M H₂SO₄ (final concentration). Confirm the Kon variants by kinetic analysis using a BIAcore biosensor. The variants screened in this way should have a higher Kon than that of the parental antibody.

### Example 4

### Construction, Screening and Characterization of Combinatorial Mutations

Single-mutation clones, isolated with confirmed desired affinity improvements, can be DNA sequenced. Beneficial mutations from six focused CDR libraries can be tabulated. A combinatorial library is constructed by multiple-site hybridization mutagenesis (see Perlak F. J. Nucleic Acids Res. 18 7457-7458 (1990), herein incorporated by reference) using degenerate oligonucleotides encoding beneficiall mutations and the parental uridinylated template. Because not all mutations may be compatible with each other, the wild-type residue should always be included at each combinatorial CDR position in each degenerate oligonucleotide. The library may be randomly sequenced to analyze the distribution of combined mutations. Assays well-known to those of skill in the art may be used to screen the combinatorial library. Once the improvement of the identified combinatorial clones are confirmed, the clones can be DNA sequenced. Then, Fab fragments can be purified or intact antibodies can be generated for subsequent functional characterizations.

### Example 5a

### Biodistribution Studies in Mice

Biodistribution studies may be performed as described previously (see Adams G. P., et al., Br. J. Cancer 77: 1405-1412 (1998), herein incorporated by reference). Briefly, tumor cell line cells in log phase may be implanted s.c. on the abdomens of 4-6 week old inbred C.B17/lcr-scid mice. After several weeks, allowing for tumor growth to about 100 mg in size, Lugol's solution can be placed in the drinking water to block thyroid accumulation of radioiodine. Allow about 48 hours to pass, 20 ug of a radio-labeled scFv (such as an I-125 iodinated antibody) is administered to cohorts of several mice by tail vein injections. Injected doses may be determined by counting the mice on a Series 30 multichannel analyzer/probe system. After sufficient time, for example 24 hours, the mice may be sacrificed and their tumors, organs and blood retentions may be determined (see id).

The specific tumor retention is measured by calculating the injected dose of labeled antibody per gram of tissue or per volume of blood retentate. These results may be plotted by the log affinity or Kon kinetic of antibody injected to compare the quantity of antibody localizing in the tumor between the varying affinities or Kon kinetic of antibodies used in the experiment.

The specific tumor retention, as measured by a percent of the injected dose per gram of tumor (ID/gram) can be at least 0.2%, at least 0.5%, at least 1.0%, at least 1.5%, at least 3%, at least 5%, at least 10%.

### Example 6a

### Tumor Penetration Studies

Immunohistochemistry studies may be utilized to determine the impact of Kon, affinity or Koff on the tumor penetration or intra-tumoral diffusion of anti-tumor antibodies in a mouse model. Briefly, 100 ug of antibody samples of varying kinetics may be administered by i.v. injection into groups of three scid mice bearing tumors expressing the appropriate tumor antigen recognized by the antibodies tested. Tumors may be excised 24 hours later and fixed in formalin, and sections of the tumors can be evaluated by immunohistochemical methods, well-known to those of skill in the art. In this way, the tumor penetrance or intra-tumoral diffusion of each type of antibody variant tested may be visually confirmed. This result can be quantified by the number of cell diameters cell staining is observed in the tumor sections.

The amount of intra-tumoral diffusion can be greater than 3, 5, 10, 20, 30, 40, 50, 60, 70, 80, 90 or 100 tumor cell diameters as measured by immunohistochemistry (IHC).

### Example 6b

### Tumor Penetration Studies

All antibodies studied had either mouse or humanized V-domains and human IgG1 constant and kappa constant domains. A panel of 6 anti-EphA2 antibodies (G5, 3F2, 7A9, 4H5s, 3D10, 4H5c and one negative control antibody (R347)) were selected for the study. The anti-EphA2 antibodies above, all specifically bind to the human EphA2 antigen. All six antibodies differ from each other in their Kₒₙ and K_{off} rates and have either similar or widely differing dissociation constants (K_{D}) as shown in Table 1. By testing such a panel of available anti-tumor antibodies, it was possible to affirm the association between an antibody having a uniform tumor penetrance with having a higher or faster Kₒₙ rate as well as having a smaller or faster K_{off} rate, rather than relying on high affinity or a slow K_{off} alone.

Two 4 week old Nude mice were injected subcutaneously on the back with approximately 5×10⁶ MDA 231KC cells in PBS. MDA 231KC cells are tumorigenic in nude mice and express human EphA2 antigen on their cell surface—test anti-EphA2 antibodies have been shown to bind the MDA 231KC cells by FACS. After 7 days when mice developed subcutaneous tumors of approximately 150 mm³ groups of mice were treated with each of the 7 antibodies described above at 10 mg/kg intravenously. Tumors were harvested at time 30 minutes, 60 minutes, 2 hours, 8 hours, 16 hours, 24 hours, 36 hours and 48 hours after subcutaneous administration and were preserved by freezing. Cryo-sections of tumors were made and stained for human IgG using an anti-human Fc antibody conjugated to HRP. The stained sections were examined microscopically to understand the distribution pattern of the antibodies into the tumors. The tumor sections were scored for the quality of the IHC staining distribution as, going from a deep tumor penetrance to least penetrance: “uniform”, “nearly uniform”, “patchy” or “weak”. Table 2 demonstrates the results of the study for each antibody tested.

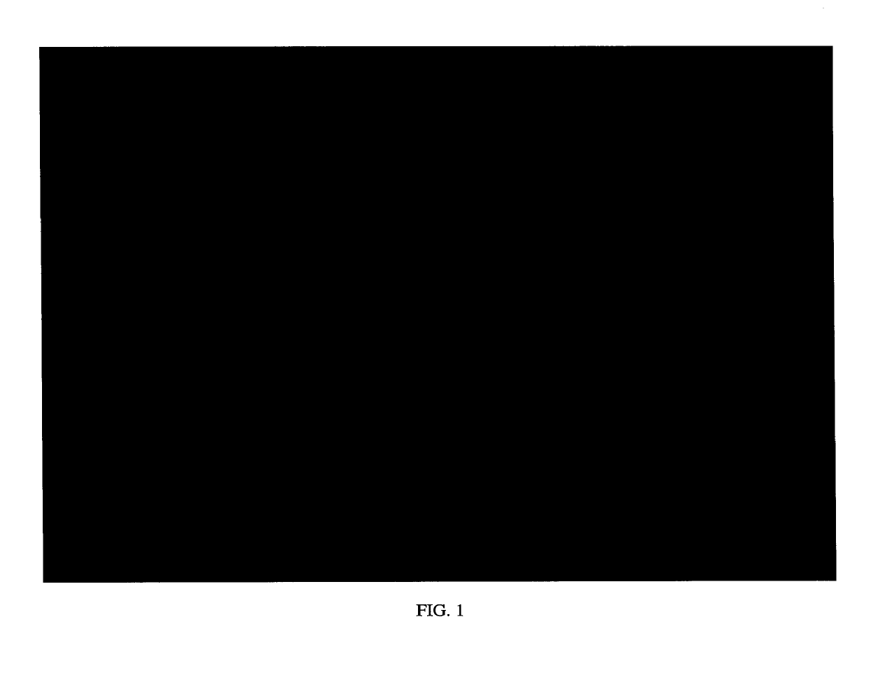

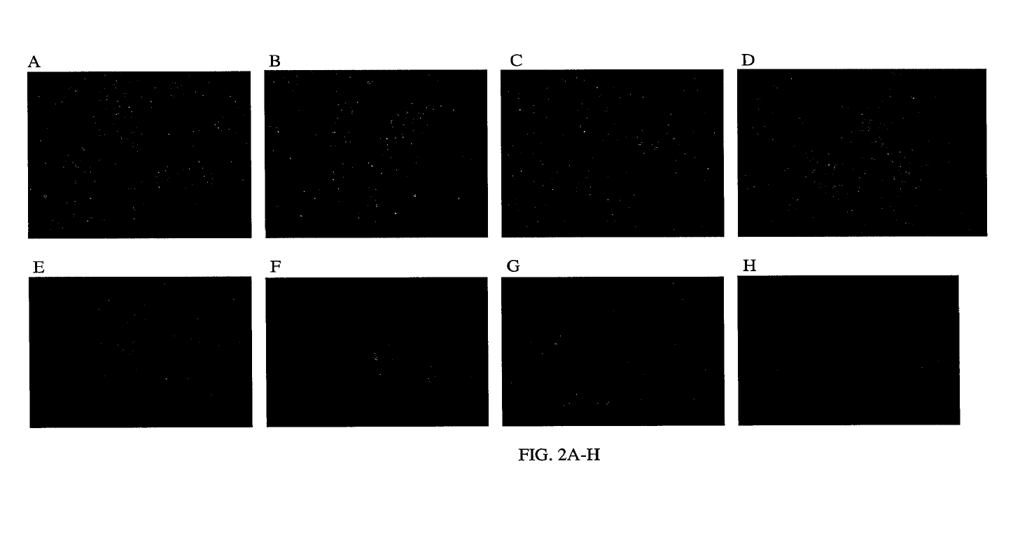

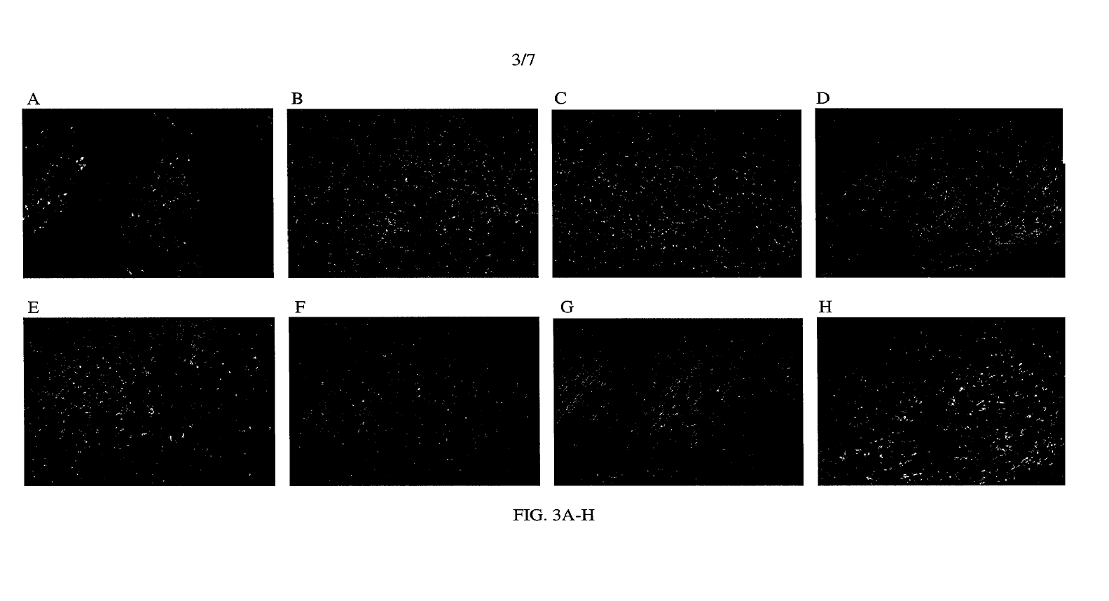

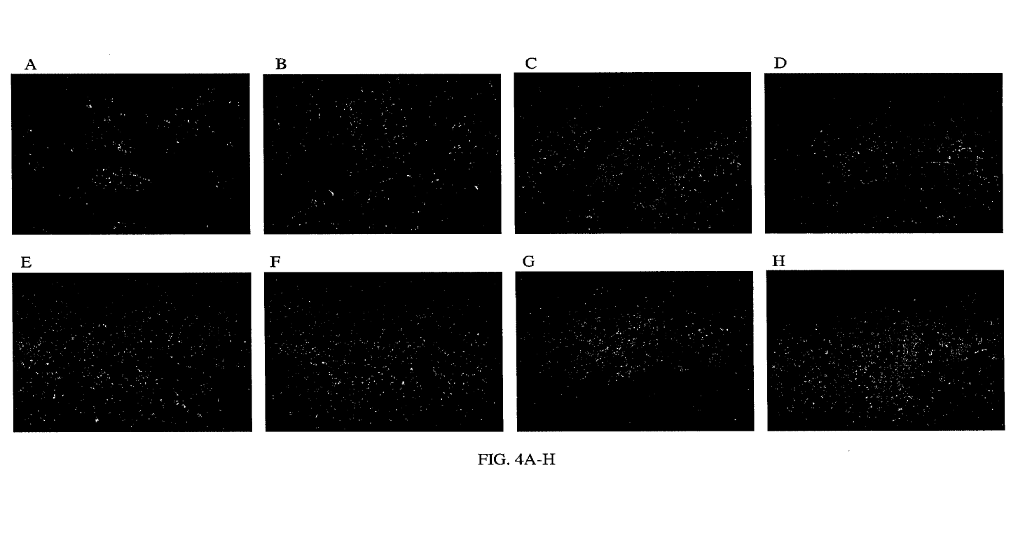

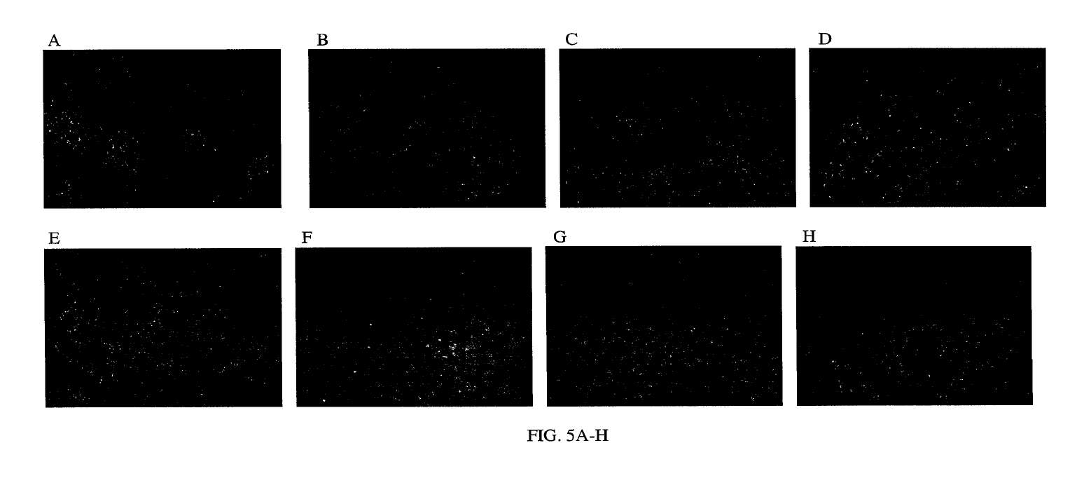

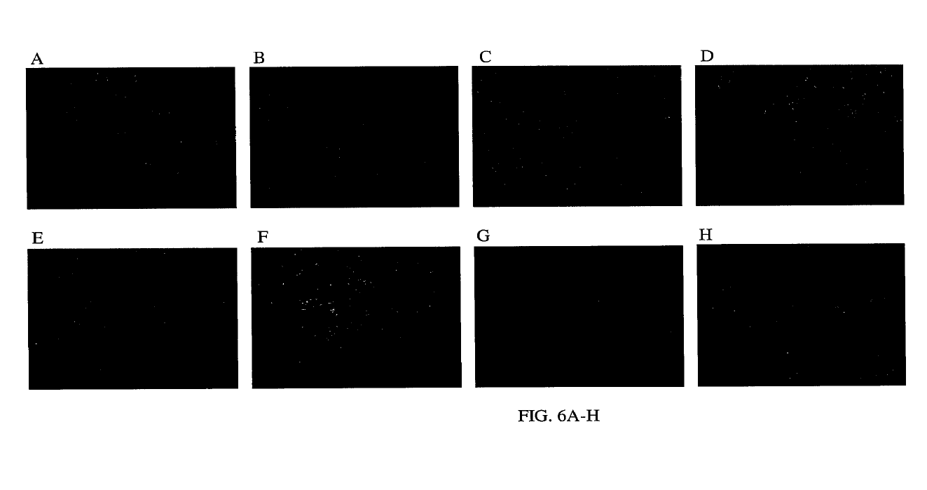

TABLE 1
Kinetic parameters of tested anti-EphA2 antibodies

| Antibody Name | Kₒₙ (M⁻¹s⁻¹) | K_{off} (s⁻¹) | K_{D} (pM) | Comments |
|---|---|---|---|---|
| R347 | | | | Negative control antibody. Does not bind human EphA2. (see FIG. 1A-H) |
| G5 | 5.32 × 10⁴ | 3.76 × 10⁻⁵ | 706 | Considered as the reference anti-EphA2 antibody (see FIG. 2A-H) |
| 3F2 | 1.89 × 10⁵ | 1.27 × 10⁻⁵ | 671 | Overall affinity is similar to the G5 reference. |
| | | | | About 3.5 fold faster Kₒₙ and about 3 fold faster K_{off} as |
| | | | | compared to G5 (see FIG. 3A-H). |
| 7A9 | 1.3 × 10⁵ | 7.4 × 10⁻⁴ | 570 | Overall affinity is similar to the G5 reference. |
| | | | | About a 2-fold faster Kₒₙ and about a 20-fold faster K_{off} as |
| | | | | compared to G5 (see FIG. 4A-H). |
| 4H5 | 9.8 × 10⁵ | 6.6 × 10⁻⁵ | 67 | About a 18-fold higher Kₒₙ and about a 2-fold faster K_{off} as |
| (shuffled) | | | | compared to G5 (see FIG. 5A-H). |
| 3D10 | 3.7 × 10⁵ | 3.6 × 10⁻⁵ | 97 | Similar K_{off} but about a 7-fold higher Kₒₙ as compared to |
| | | | | G5 (see FIG. 6A-H). |
| 4H5 | 7.5 × 10⁵ | 1.05 × 10⁻³ | 1400 | About a 14-fold faster Kₒₙ and about a 28-fold faster K_{off} as |
| (CDR corrected) | | | | compared to G5 (see FIG. 7A-H). |

TABLE 2
Kₒₙ influences IgG tumor penetrance

| Antibody | 30′ | 1 hr. | 2 hr. | 8 hr. | 16 hr. |
|---|---|---|---|---|---|
| R347 | Immediate | Immediate | Immediate | Immediate | Immediate |
| Control IgG | perivascular space | perivascular space | perivascular space | perivascular space | perivascular space |
| G5 Reference | Excellent distribution | Nearly uniform | Uniform | Nearly uniform | Patchy |
| EphA2 antibody | in broad cords. 50% | distribution. 90% | distribution. 90% | distribution. 90% | staining. 60% |
| | staining. Some | staining. Some | staining. Mostly | staining. Some | staining. Some |
| | internalized. | internalized. | internalized | internalized | internalized |
| 3F2 | Excellent distribution | Uniform | Uniform | Uniform | 60% |
| | in broad cords. 60% | staining. 100% | staining. ~90% | staining. ~80% | staining |
| | staining. Some | staining. 50% | staining. 50% | staining. 20% | patchy. Rare |
| | internalized. | internalized. | internalized. | internalized. | internalization. |
| 7A9 | Deep | Deep | Nearly uniform | Nearly uniform | Nearly uniform |
| | distribution in | distribution in | distribution. 80% | distribution. 80% | distribution. 80% |
| | broad cords. 50% | broad cords. 70% | staining. 50% | staining. 50% | staining. 30% |
| | staining. | staining. Some | internalized. | internalized. | internalized. |
| | | internalized. | | | |
| 4H5 | Deep distribution | Deep | Nearly uniform | Uniform | Uniform |
| (shuffled) | in broad cords. 60% | distribution in | distribution. 80% | distribution. 100% | distribution. 100% |
| | staining. Some | broad cords. 60% | staining. 60% | staining. 50% | staining. 30% |
| | internalized. | staining. Some | internalized. | internalized. | internalized. |
| | | internalized. | | | |
| 3D10 | Deep distribution. | Deep | Deep | Patchy | Patchy |
| | 40% staining. | distribution. 40% | distribution. 30% | staining. 30% | staining. 40% |
| | | staining. | staining. Some | staining. | staining. Some |
| | | | internalized. | | internalized. |
| 4H5 | Very patchy. | Patchy | Patchy | Patchy | Very patchy. |
| (corrected) | Perivascular. 10% | staining. 25% | staining. 60% | staining. 60% | Perivascular. 10% |
| | staining. | staining. | staining. 50% | staining. 50% | staining. |
| | | | internalized. | internalized. | |

| Antibody | 24 hr. | 36 hr. | 48 hr. |
|---|---|---|---|
| R347 | Immediate | Immediate | Immediate |
| Control IgG | perivascular space | perivascular space | perivascular space |
| G5 Reference | Patchy | Patchy | Patchy |
| EphA2 antibody | staining. 60% | staining. 60% | staining. 60% |
| | staining. Some | staining. Some | staining. Some |
| | internalized | internalized | internalized |
| 3F2 | Uniform | 60% | Uniform |
| | staining. ~90% | staining | staining. 100% |
| | staining. 20% | patchy. Rare | staining. 50% |
| | internalized | internalization. | internalized. |
| 7A9 | Nearly uniform | Nearly uniform | Nearly uniform |
| | distribution. 70% | distribution. 70% | distribution. 100% |
| | staining. 30% | staining. 30% | staining. 30% |
| | internalized. | internalized. | internalized. |
| 4H5 | Uniform | Uniform | Uniform |
| (shuffled) | distribution. 100%. | distribution. 100% | distribution. 100% |
| | 50% | staining. 50% | staining. 20% |
| | internalized. | internalized. | internalized. |
| 3D10 | Patchy | Patchy | Patchy |
| | staining. 30% | staining. 40% | staining. 20% |
| | staining. | staining. Some | staining. Some |
| | | internalized. | internalized. |
| 4H5 | Weak staining | Weak staining | Very patchy. |
| (corrected) | overall. | overall. | Perivascular. 10%. |

Observations of tumor penetrance of the tested anti-EphA2 antibodies in Table 2: for the negative control R347 IgG, this negative control antibody does not show any penetration into the targeted tumor because it does not bind to any antigen on the tumor cells. As a result after extra-vasation, it remains confined to the perivascular space (see FIGS. 1A-H).

For the G5 anti-EphA2 reference antibody, the initial distribution is quite good but from 16 hours onward it reveals poor penetrance into the tumor (see FIGS. 2A-H).

For the 3F2 antibody, except for times 16 and 36 hours, this antibody shows a uniform penetrance into the tumor. Although 3F2′s affinity is very similar to G5, it has a 3.5 fold faster Kon rate and a 3 fold faster Koff rate, thereby facilitating its penetration into the tumor (see FIGS. 3A-H).

For the 7A9 antibody, it shows a uniform penetrance into the tumor. Although 7A9′s affinity is very similar to G5, it has a 2 fold faster Kon rate and a 20 fold faster Koff rate, thereby facilitating its penetration into the targeted tumor. This result also lends credence to the proposition that the Kon rate plays a more significant role over the Koff rate as far as penetration into tumor is concerned (see FIGS. 4A-H).

For the 4H5 shuffled antibody, it shows a uniform penetrance into the tumor. 4H5 has a nearly 10 fold higher affinity than G5, which results from a 18 fold faster Kon rate and 2 fold faster Koff rate. In spite of its very high affinity as compared to G5, 4H5 shuffled shows excellent uniform tumor penetration. (see FIGS. 5A-H).

The 3D10 antibody shows poor penetrance into the tumor. Its affinity is almost same as that of 4H5 shuffled, but differs in its Kon and Koff rates. 3D10 has nearly 10 fold higher affinity than G5, which results from a 7 fold faster Kon rate and same Koff rate. Because 3D10 can latch onto its target EphA2 on tumor cells quickly due to its fast Kon rate, but not come off easily due to its slow Koff rate, 3D10 fails to penetrate deeper after extra-vasation. As a result, penetration into the tumor is poor (see FIGS. 6A-H).

For the 4H5 corrected antibody, its low affinity precludes the antibody from remaining bound long enough to its antigen on targeted tumor cells. Because it tends to fall off easily from its target due to its low affinity (see Table 1), 4H5 corrected cannot effectively penetrate into the tumor. As a result, after the initial time points 4H5 corrected fails to penetrate into tumor (see FIGS. 7A-H).

The results of the study indicate that unlike what has been reported in the literature, it is not the K_{D} or dissociation constant that determines the efficacy of an antibody to penetrate solid tumors. In fact it is the Kₒₙ and K_{off} rates that play a major role in determining if an antibody can cross the tumor's affinity barrier after extra-vasation. Antibodies with a higher or faster Kₒₙ and faster K_{off} are able to penetrate deeper into tumors than antibodies with similar K_{D} but having slower Kₒₙ and slower K_{off} rates. Therefore, during super-optimization of antibodies for affinity it is important that more emphasis be placed on the Kₒₙ and K_{off} rates than on the overall K_{D}. For example, in the optimization of agonistic antibodies that bind to tumor cells and send an apoptotic signal, it will be desirable to have or engineer antibody variants with faster Kₒₙ and faster K_{off} rates such that the improved anti-tumor antibody specifically binds to its targeted tumor cells quickly, transmits its death signal utilizing its effector functioning, and then comes off the tumor cell to be able to subsequently bind another tumor cell to kill it.

### Equivalents

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

1. A method of improving a recombinant anti-tumor antibody's penetrance into a targeted tumor, comprising:
(a) increasing the Kₒₙ rate by at least 2 fold as compared to the same recombinant anti-tumor antibody without such an increase in Kₒₙ rate; and
(b) achieving a uniform tumor penetrance by said improved recombinant anti-tumor antibody into said targeted tumor as measured by immunohistochemistry staining (IHC) as compared to the same recombinant anti-tumor antibody without such an increase in Kₒₙ rate and demonstrates a non-uniform or patchy staining as measured by IHC.

2. The method of claim 1, wherein said method further comprises after step (a), a step of increasing the K_{off} rate by at least 2 fold as compared to the same recombinant anti-tumor antibody without such an increase in K_{off} rate.

3.-4. (canceled)

5. The method of claim 1, wherein said uniform tumor penetrance by said improved recombinant anti-tumor antibody into said targeted tumor demonstrates at least 70% IHC staining.

6.-8. (canceled)

9. A method of improving a recombinant anti-tumor antibody's penetrance into a targeted tumor, comprising:
(a) increasing the Kₒₙ rate by at least 2 fold as compared to the same recombinant anti-tumor antibody without such an increase in Kₒₙ rate; and
(b) achieving an intra-tumoral diffusion greater than 3 tumor cell diameters as measured by IHC, as compared to the same recombinant anti-tumor antibody without such an increase in Kₒₙ rate and demonstrates an intra-tumoral diffusion of 3 or less tumor cell diameters as measured by IHC.

10. (canceled)

11. The method of claim 1, wherein said improved recombinant anti-tumor antibody specifically binds to targets CD20, HER2/neu, VEGF, EphA2, integrin αᵥβ3, EphA4, CD2, CD19, CD22, cMET, ALK, DLL4, or CEA.

12. The method of claim 9, wherein said improved recombinant anti-tumor antibody specifically binds to targets CD20, HER2/neu, VEGF, EphA2, integrin aᵥβ3, EphA4, CD2, CD19, CD22, cMET, ALK, DLL4, or CEA.

13. The method of claim 1, wherein said improved recombinant anti-tumor antibody has a Kₒₙ rate of at least 10⁴ M⁻¹s⁻¹ or greater.

14. The method of claim 9, wherein said improved recombinant anti-tumor antibody has a Kₒₙ rate of at least 10⁴ M⁻¹s⁻¹ or greater.

15. The method of claim 1, wherein said improved recombinant anti-tumor antibody has a K_{off} rate of 10⁻³ s⁻¹ or less.

16. The method of claim 9, wherein said improved recombinant anti-tumor antibody has a K_{off} rate of 10⁻³ s⁻¹ or less.

17.-22. (canceled)

23. A recombinant anti-tumor antibody having improved tumor penetrance into a targeted tumor, produced by a method comprising:
(a) increasing the Kₒₙ rate by at least 2 fold as compared to the same recombinant anti-tumor antibody without such an increase in Kₒₙ rate; and
(b) achieving a uniform tumor penetrance by said improved recombinant anti-tumor antibody into said targeted tumor as measured by immunohistochemistry staining (IHC) as compared to the same recombinant anti-tumor antibody without such an increase in Kₒₙ rate and demonstrates a non-uniform or patchy staining as measured by IHC.

24. A recombinant anti-tumor antibody having improved tumor penetrance into a targeted tumor, produced by a method comprising:
(a) increasing the Kₒₙ rate by at least 2 fold as compared to the same recombinant anti-tumor antibody without such an increase in Kₒₙ rate; and
(b) achieving an intra-tumoral diffusion greater than 3 tumor cell diameters as measured by IHC, as compared to the same recombinant anti-tumor antibody without such an increase in Kₒₙ rate and demonstrates an intra-tumoral diffusion of 3 or less tumor cell diameters as measured by IHC.

25.-50. (canceled)

51. A composition comprising, the recombinant anti-tumor antibody having improved tumor penetrance of claim 23, and a pharmacologically acceptable diluent or excipient.

52. A method of treating a tumor in a patient in need thereof, comprising administering a therapeutically effective amount of the composition of claim 51.

53. A composition comprising, the recombinant anti-tumor antibody having improved tumor penetrance of claims 24, and a pharmacologically acceptable diluent or excipient.

54. A method of treating a tumor in a patient in need thereof, comprising administering a therapeutically effective amount of the composition of claim 53.
